(19) United States
(12) Patent Application Publication
(10) Pub. No.: US 2021/0113798 A1
(43) Pub. Date: Apr. 22, 2021

(54) Connector

(71) Applicant: TELEFLEX LIFE SCIENCES PTE. LTD., Hamilton (SG)

(72) Inventors: Wee Kheong OOI, Singapore (SG); Xiang Long ZHANG, Tianjin (CN); Shan LIU, Tianjin (CN)

(21) Appl. No.: 17/134,323

(22) Filed: Dec. 26, 2020

Related U.S. Application Data
(63) Continuation of application No. PCT/EP2019/066863, filed on Jun. 25, 2019.

(30) Foreign Application Priority Data
Jun. 26, 2018 (GB) 1810485.1

(51) Int. Cl. A61M 16/04; A61M 16/08

(52) U.S. Cl. CPC A61M 16/0434; A61M 16/0402; A61M 16/0816

(57) ABSTRACT

A removable connector is provided for an artificial airway device, having an airway tube, a cuff and an inflation tube for selectively inflating and/or deflating the cuff. The connector includes a conduit which may be inserted into a lumen of the airway tube, the connector being adapted to be removable and replaceable from the lumen of the airway tube.

### CROSS-REFERENCE TO RELATED APPLICATIONS

This patent application is a continuation of International Patent Application PCT/EP2019/066863, filed on Jun. 25, 2019, which claims priority to United Kingdom Patent Application no. 1810485.1, filed Jun. 26, 2018, the disclosures of which are incorporated herein by reference in their entireties.

### FIELD OF THE INVENTION

The present invention relates to a connector, and in particular to a connector for a laryngeal mask airway device.

### BACKGROUND

Artificial airway devices such as the laryngeal mask airway device are well known devices useful for establishing airways in unconscious patients. In its most basic form a laryngeal mask airway device consists of an airway tube and a mask carried at one end of the airway tube, the mask having a peripheral formation often known as a “cuff” which is capable of conforming to and of fitting within, the actual and potential space behind the larynx of the patient so as to form a seal around the laryngeal inlet. The cuff can be inflatable, and in most variants it surrounds a hollow interior space or lumen of the mask, the at least one airway tube opening into the lumen. U.S. Pat. No. 4,509,514 is one of the many publications that describe laryngeal mask airway devices. Such devices have been in use for many years and offer an alternative to the older, even better known endotracheal tube. For at least seventy years, endotracheal tubes comprising a long slender tube with an inflatable balloon disposed at the tube's distal end have been used for establishing airways in unconscious patients. In operation, the endotracheal tube's distal end is inserted through the mouth of the patient, past the patient's trachea. Once so positioned, the balloon is inflated so as to form a seal with the interior lining of the trachea. After this seal is established, positive pressure may be applied to the tube's proximal end to ventilate the patient's lungs. Also, the seal between the balloon and the inner lining of the trachea protects the lungs from aspiration (e.g., the seal prevents material regurgitated from the stomach from being aspirated into the patient's lungs).

Although they have been successful, endotracheal tubes suffer from several major disadvantages. The principal disadvantage of the endotracheal tube relates to the difficulty of properly inserting the tube. Inserting an endotracheal tube into a patient is a procedure that requires a high degree of skill. Also, even for skilled practitioners, insertion of an endotracheal tube is sometimes difficult or not possible. In many instances, the difficulty of inserting endotracheal tubes has tragically led to the death of a patient because it was not possible to establish an airway in the patient with sufficient rapidity. Also, inserting an endotracheal tube normally requires manipulation of the patient's head and neck and further requires the patient's jaw to be forcibly opened widely. These necessary manipulations make it difficult, or undesirable, to insert an endotracheal tube into a patient who may be suffering from a neck injury.

In contrast to the endotracheal tube, it is relatively easy to insert a laryngeal mask airway device into a patient and thereby establish an airway. Also, the laryngeal mask airway device is a “forgiving” device in that even if it is inserted improperly, it still tends to establish an airway. Accordingly, the laryngeal mask airway device is often thought of as a “life saving” device. Also, the laryngeal mask airway device may be inserted with only relatively minor manipulation of the patient's head, neck and jaw. Further, the laryngeal mask airway device provides ventilation of the patient's lungs without requiring contact with the sensitive inner lining of the trachea and the internal diameter of the airway tube is typically significantly larger than that of the endotracheal tube. Also, the laryngeal mask airway device does not interfere with coughing to the same extent as endotracheal tubes. Largely due to these advantages, the laryngeal mask airway device has enjoyed increasing popularity in recent years.

U.S. Pat. No. 4,509,514 describes a laryngeal mask airway device which consists of the basic parts which make up most if not all laryngeal mask airway devices, namely an airway tube opening at one end into the interior of a hollow mask portion shaped to fit readily behind the larynx of a patient. The periphery of the mask is formed by a cuff which in use forms a seal around the opening of the larynx. This enables the airway to be established effectively.

Laryngeal mask airway devices with specific provision for gastric-discharge drainage have been developed, as exemplified by U.S. Pat. No. 4,995,388 (FIGS. 7 to 10); U.S. Pat. Nos. 5,241,956; and 5,355,879. These devices generally incorporate a small-diameter drainage tube having an end located at the distal end of the mask, so as to lie against the upper end of the upper oesophageal sphincter when the mask is in place, the tube being of sufficient length to extend out of the mouth of the patient to enable active or passive removal of gastric discharge from the upper oesophageal sphincter. According to alternative proposals, the drainage tube may extend beyond the distal end of the mask, into the oesophagus itself (U.S. Pat. No. 4,995,388, FIGS. 7 and 11).

Laryngeal mask airway devices are now commonly used to aid in insertion of endotracheal tubes, and such devices are referred to as intubating laryngeal masks, an example being Applicant's own “Fastrach”™ device.

One of the problems associated with some laryngeal mask airway devices is that the connector is typically attached to the device and may not be removed therefrom. Thus, if the connector is to be changed or replaced, the entire laryngeal mask airway device must be changed which may take additional time and/or reduce the flexibility available to the clinician during the medical procedure.

Another problem associated with some laryngeal mask airway devices is that if the connector is removed from the airway device, there is nothing to secure the connector to the airway device such that the connector may be lost during a clinical procedure. During clinical procedures it is important that small objects such as connectors don't get lost, since these may be required to be accessed quickly during the procedure.

### SUMMARY OF THE INVENTION

The present invention seeks to ameliorate problems associated with the prior-art described above.

According to a first aspect of the invention there is provided a removable connector for use with an artificial airway device comprising an airway tube, a cuff and an inflation tube for selectively inflating and/or deflating the cuff, the connector comprising a conduit which may be inserted into a lumen of the airway tube, the connector being adapted to be removable and replaceable from the lumen of the airway tube.

Preferably, the conduit of the connector may be adapted to enable the connector to be removed from the lumen of the airway tube. Advantageously, the provision of a removable connector provides the clinician with greater flexibility during a medical procedure. Advantageously, the removal of the conduit of the connector from the airway device allows another connector to be connected to the airway tube, and/or allows for manipulation of the airway tube and/or insertion of another device through the airway tube. Preferably, the connector may be used to connect the airway tube of the airway device to an air supply.

It is preferred that the conduit of the connector may be adapted for removal from the lumen of the airway tube by having a plurality of external circumferential friction reducing formations which may cooperate with the internal surface of the lumen of the airway tube. In one embodiment, the internal surface of the lumen of the airway tube may comprise a plurality of circumferential friction reducing formations which may cooperate with the external surface of the conduit. In one embodiment, the conduit of the connector may have a smooth surface. In an alternative embodiment, the external surface of the conduit may comprise a screw thread which cooperates with a corresponding thread on the internal surface of the airway tube lumen. In another embodiment, the external surface of the conduit may cooperate with the internal surface of the airway tube lumen by means of a push fit or friction fit with airway tube. Advantageously, the friction reducing formations and/or other cooperating means and/or friction fit between the external surface of the conduit of the connector and the internal surface of the airway tube lumen allows the conduit of the connector to be securely held in place within the airway tube when the device is in use, and allows for removal of the connector from the airway tube when required.

It is preferred that the conduit of the connector may be adapted for removal from a preformed connector section, preferably provided at the proximal end of the airway tube. Preferably, the preformed connector section of the airway tube is dimensioned to accommodate the connector. Preferably, the preformed connector section has a wider diameter than the diameter at the distal section of the airway tube.

Preferably, the distal end of the airway tube has a substantially circular diameter, wherein the thickness of the walls at the top and bottom of the distal end of the airway tube is substantially the same as the thickness of the walls at the left and right of the distal end of the airway tube.

Preferably, the preformed connector section of the airway tube has a substantially oval shape. Preferably, the proximal end of the preformed connector section of the airway tube has a substantially oval shape. Preferably, the thickness of the walls at the top and bottom of the preformed connector section of the airway tube is greater than the walls at the left and right of the preformed connector section of the airway tube. Advantageously, the substantially oval shape of the preformed connector section of the airway tube assists in holding the connector securely within the airway tube, whilst allowing the connector to removed from the airway tube if required.

Preferably, the internal surface of the preformed connector section of the airway tube comprises a plurality of friction reducing formations that may cooperate with the external surface of the conduit. Advantageously, this allows the connector to be securely held in place within the airway tube when the device is in use, and allows for removal of the connector from the airway tube when required.

Preferably, the inflation tube is connected to the cuff of the device.

Preferably, the connector comprises a conduit having a circumferential wall defining a lumen which may be inserted into the lumen of the airway tube. Preferably, the connector comprises a connector plate which surrounds the conduit. Preferably, the connector plate extends from the conduit at an angle substantially perpendicular to the axis of the conduit.

Preferably, the connector comprises means for tethering the connector to an artificial airway device. Preferably, the connector comprises means for tethering the connector to the airway tube of an artificial airway device.

Preferably, the means for tethering the connector to the airway device comprises the inflation tube which engages with a portion of the connector.

Preferably, the connector can move freely along the length of the inflation tube. Preferably, the inflation tube has a length which assists in movement of the conduit along the length of the inflation tube, for example, when the conduit has been removed from the device. Preferably, the means for tethering assists in the connector moving freely along the length of the inflation tube. Advantageously, this allows for greater flexibility during removal of the connector from the airway tube.

In one embodiment, the means for tethering the inflation tube to the connector holds the inflation tube captive such that it is not detachable from the connector. Advantageously, this embodiment prevents the connector from being inadvertently detached from the inflation tube after being removed from the airway tube.

Preferably, the connector plate comprises means for holding the inflation tube such that the connector is tethered to the airway device.

Preferably, the means for tethering comprises at least one aperture provided within the connector plate. Preferably, the at least one aperture is a closed aperture that is provided within the connector plate. Preferably, the inflation tube is held within the closed aperture.

In this embodiment, the at least one aperture is closed such that the inflation tube is tethered to and held captive by the connector, such that the inflation tube cannot be removed from the connector.

In another embodiment, the means for tethering allows for the attachment and/or detachment of the inflation tube from the connector. Advantageously, this embodiment provides flexibility for the clinician if they need to use the connector during a medical procedure, as the tether can be reversibly attached and/or detached.

In one embodiment, the means for tethering comprises at least one aperture that is open at one portion thereof. Preferably, the at least one open aperture forms a clip such that the inflation tube may be selectively attached and/or detached from the connector. Preferably, the at least one open aperture is provided on the connector plate. Preferably, the clip allows for the attachment and/or detachment of the inflation tube from the connector. Preferably, clip is provided on the connector. Preferably, the clip is provided on the connector plate.

Preferably, the diameter of the at least one open or closed aperture provided on the connector and the diameter of the inflation tube are configured such that the inflation tube can freely slide within the at least one open or closed aperture. Preferably, the diameter of the at least one open or closed aperture on the connector and the dimensions of the inflation tube are configured such that the inflation tube can freely slide within the at least one open or closed aperture. Preferably, the diameter of the open or closed aperture is between around 0.4 and 0.5 mm. Preferably, the diameter of the inflation tube is between around 0.2 and 0.3 mm.

Preferably, the connector plate may comprise a recess. Preferably, the recess is provided on a side of the connector plate that is opposite to the aperture. In one embodiment, the recess may be provided on a side of the connector plate that is adjacent to the aperture. Preferably, the recess is provided in the form of a cut away portion having three substantially straight sides. Advantageously, the recess may assist in removal of the connector from the airway tube.

Typically, the cuff is inflatable. Preferably, the inflation tube is attached to means for indicating inflation and/or means for monitoring and/or controlling the pressure within the cuff. In one embodiment, the inflation tube is attached to a one way valve, as known in the prior art. In another embodiment, the cuff may be non-inflatable.

Preferably, the inflation tube includes a cuff pressure monitor or pilot valve that may be released from the inflation tube. Advantageously, the cuff pressure monitor or pilot valve may be released from the inflation tube without loss of cuff pressure.

Preferably, the cuff is of roughly elliptical shape and is formed with a port into which is sealed one end of an inflation tube. Preferably, the inflation tube is a flexible plastics tube.

Preferably, the connector is connected to a separate biteblock. In another embodiment, the connector comprises an integral biteblock. Preferably, the conduit of the connector may comprise a biteblock.

Preferably, the conduit of the connector (i.e. the part that is inserted into an airway tube) has a constant diameter. Preferably, the diameter of the conduit of the connector is between around 0.8 and 1.2 cm. More preferably, the diameter of the conduit of the connector is around 1.0 cm.

In one embodiment, the connector comprises a plastics material. Preferably, the connector comprises PVC.

In one embodiment, the artificial airway device is a laryngeal mask airway device. In one embodiment, the artificial airway device is a pediatric laryngeal mask airway device.

According to a second aspect, there is provided an artificial airway device to facilitate lung ventilation of a patient, comprising an airway tube including a lumen, a mask at one end of the airway tube, the mask including a backplate and having a cuff capable of forming a seal around the circumference of the laryngeal inlet, the cuff surrounding a hollow interior space or lumen of the mask and the airway tube opening into the lumen of the mask, and inflation tube for selectively inflating and/or deflating the cuff, and a removable connector disposed at the proximal end of the airway tube, the connector comprising a conduit which may be inserted into the lumen of an airway tube, the connector being adapted to be removable and replaceable from the lumen of the airway tube.

Preferably, the conduit may be adapted to enable the connector to be removed from the lumen of the airway tube. Advantageously, the provision of a removable connector provides the clinician with greater flexibility during a medical procedure. Advantageously, the removal of the conduit from the airway device allows another connector to be connected to the airway tube, and/or allows for manipulation of the airway tube and/or insertion of another device through the airway tube.

It is preferred that the conduit of the connector may be adapted for removal from the lumen of the airway tube by having a plurality of external circumferential friction reducing formations which may cooperate with the internal surface of the lumen of the airway tube. In one embodiment, the internal surface of the lumen of the airway tube may comprise a plurality of circumferential friction reducing formations which may cooperate with the external surface of the conduit. In one embodiment, the external surface of the conduit of the connector is smooth. In an alternative embodiment, the external surface of the conduit of the connector may comprise a screw thread which cooperated with a corresponding thread on the internal surface of the airway tube lumen. In another embodiment, the external surface of the conduit of the connector may cooperate with the internal surface of the airway tube lumen by means of a push fit or friction fit with airway tube. Advantageously, the friction reducing formations and/or other cooperating means and/or friction fit between the external surface of the conduit of the connector and the internal surface of the airway tube lumen allows the conduit of the connector to be securely held in place within the airway tube when the device is in use, and allows for removal of the connector from the airway tube when required.

It is preferred that the conduit of the connector may be adapted for removal from a preformed connector section provided at the proximal end of the airway tube. Preferably, the preformed connector section of the airway tube is dimensioned to accommodate the connector. Preferably, the preformed connector section has a wider diameter than the diameter at the distal section of the airway tube.

Preferably, the distal end of the airway tube has a substantially circular diameter, wherein the thickness of the walls at the top and bottom of the distal end of the airway tube is substantially the same as the thickness of the walls at the left and right of the distal end of the airway tube.

Preferably, the internal surface of the preformed connector section of the airway tube comprises a plurality of friction reducing formations that may cooperate with the external surface of a conduit of a connector. Advantageously, this allows the conduit to be securely held in place within the airway tube when the device is in use, and allows for removal of the connector from the airway tube when required.

Preferably, the preformed connector section of the airway tube has a substantially oval shape. Preferably, the thickness of the walls at the top and bottom of the preformed connector section of the airway tube is greater than the walls at the left and right of the preformed connector section of the airway tube. Advantageously, the oval shape of the preformed connector section of the airway tube assists in holding the connector securely within the airway tube, whilst allowing the connector to removed from the airway tube if required.

Preferably, the connector comprises means for tethering the connector to the artificial airway device. Preferably, the means for tethering comprises an inflation tube which engages with a portion of the connector.

Preferably, the connector can move freely along the length of the inflation tube. Preferably, the inflation tube has a length that assists in the movement of the connector along the inflation tube. Preferably, the means for tethering allows the connector to move freely along the length of the inflation tube. Advantageously, this allows for greater flexibility during removal of the connector from the airway tube.

In one embodiment, the means for tethering holds the inflation tube captive such that it is not detachable from the connector. Advantageously, this embodiment prevents the connector from being inadvertently detached from the inflation tube after being removed from the airway tube. In this embodiment, the connector remains attached to the inflation tube after removal of the connector from the airway tube.

Preferably, the connector comprises a conduit having a circumferential wall defining a lumen which may be inserted into the lumen of the airway tube. Preferably, the connector comprises a connector plate which surrounds the conduit. Preferably, the connector plate extends from the conduit at an angle substantially perpendicular to the axis of the conduit. Preferably, the connector plate comprises means for tethering the inflation tube.

Preferably, the means for tethering comprises at least one aperture provided within the connector plate. Preferably, the at least one aperture is a closed aperture that is provided within the material of the connector plate. In this embodiment, the at least one aperture is closed such that the inflation tube is tethered to and held captive by the connector, such that the inflation tube cannot be removed from the connector.

In another embodiment, the means for tethering allows for attachment and/or detachment of the inflation tube from the connector. Advantageously, this embodiment provides flexibility for the clinician if they need to use the connector during a medical procedure. In this embodiment, the connector can be removed if no longer needed with the device (for example, if a different connector is used).

Preferably, the means for tethering comprises a clip that allows for the attachment and/or detachment of the inflation tube from the connector. Preferably, the connector comprises a clip that allows for the attachment and/or detachment of the inflation tube from the connector. Preferably, the clip is provided on the connector plate. In one embodiment, the means for tethering comprises at least one aperture that is open at one portion thereof. Preferably, the at least one open aperture forms the clip such that the inflation tube may be selectively attached and/or detached from the connector. Preferably, the at least one open aperture is provided on the connector plate. In this embodiment, the connector may clip to the inflation tube, allowing for attachment and detachment of the inflation tube from the connector.

Preferably, the diameter of the at least one open or closed aperture on the connector and the diameter of the inflation tube are configured such that the inflation tube can freely slide within the at least one open or closed aperture. Preferably, the diameter of the at least one open or closed aperture on the connector and the dimensions of the inflation tube are configured such that the inflation tube can freely slide within the at least one open or closed aperture. Preferably, the diameter of the open or closed aperture is between around 0.4 and 0.5 mm. Preferably, the diameter of the inflation tube is between around 0.2 and 0.3 mm.

Preferably, the connector plate may comprise a recess. Preferably, the recess is provided on a side of the connector plate that is opposite to the aperture. In one embodiment, the recess may be provided on a side of the connector plate that is adjacent to the aperture. Preferably, the recess is provided in the form of a cut away portion having three substantially straight sides. Advantageously, the recess may assist in removal of the connector from the airway tube.

Typically, the cuff is inflatable. Preferably, the inflation tube is attached to means for monitoring and/or controlling the pressure within the cuff. In one embodiment, the inflation tube is connected to a one way valve, as is known in the prior art. In one embodiment, the cuff may be non-inflatable.

Preferably, the inflation tube includes a cuff pressure monitor or pilot valve that may be released from the inflation tube. Advantageously, the cuff pressure monitor or pilot valve may be released from the inflation tube without loss of cuff pressure.

Preferably, the device comprises a biteblock that is separate to the connector. In another embodiment, the connector comprises a biteblock that is integral with the connector. Preferably, the conduit of the connector may comprise a biteblock

Preferably, the conduit of the connector (i.e. the part that is inserted into an airway tube) has a constant diameter. Preferably, the diameter of the conduit of the connector is between around 0.8 and 1.2 cm. More preferably, the diameter of the conduit of the connector is around 1.0 cm.

Preferably, the artificial airway device comprises a plastics material, such as PVC and/or silicone. Preferably, the connector comprises PVC. Preferably, the inflation tube comprises silicone.

According to a third aspect, there is provided a method of using an artificial airway device comprising a removable connector.

Preferably, the method comprises the step of tethering the connector to an inflation tube.

Preferably, there is provided a method of using an artificial airway device comprising a removable connector, wherein the connector is tethered to an inflation tube.

Preferably, the method comprises using an artificial airway device according to the second aspect of the invention comprising a removable connector according to the first aspect of the invention, wherein the connector is tethered to an inflation tube.

According to a fourth aspect, there is provided a method of treating a patient using an artificial airway device according to the second aspect of the invention.

### BRIEF DESCRIPTION OF THE DRAWINGS

The invention will now be further described by way of example and with reference to the accompanying drawings, in which:

FIG. 1 is a side view of an artificial airway device and a removable connector according to an embodiment of the invention;

FIG. 2 is a front side view of an artificial airway device and a removable connector according to an embodiment of the invention;

FIG. 3a is a schematic view of a removable connector according to one embodiment of the invention;

FIG. 3b is a schematic view of a removable connector in combination with an inflation tube according to one embodiment of the invention;

FIG. 4a is a schematic view of a removable connector according to another embodiment of the invention;

FIG. 4b is a schematic view of a removable connector in combination with an inflation tube according to an embodiment of the invention;

FIG. 5a is a top side view of a removable connector according to the embodiment shown in FIG. 3a;

FIG. 5b is a top side view of a removable connector according to the embodiment shown in FIG. 4a;

FIG. 6a is a side view of a removable connector in combination with an airway tube and an inflation tube according to an embodiment of the invention;

FIG. 6b is a side view of a removable connector in combination with an inflation tube according to an embodiment of the invention, wherein the connector has been removed from the airway tube;

FIG. 7a is a side view of a removable connector in combination with an inflation tube, according to an embodiment of the invention;

FIG. 7b is a front view of a removable connector according to an embodiment of the invention, wherein the comlector has been removed from the inflation tube;

FIG. 8 is a perspective end view of a portion of the airway tube that may be used with a connector according to an embodiment of the invention; and

FIG. 9 is an end view of a removable connector according to an embodiment of the invention.

Referring to the drawings, there is provided a removable connector 2 for use with an artificial airway device 4 comprising an airway tube 6, a cuff 8 and an inflation tube 10 for selectively inflating and/or deflating the cuff 8, the connector 2 comprising a conduit 12 which may be inserted into a lumen 14 of the airway tube 6, the connector 2 being adapted to be removable and replaceable from the lumen 14 of the airway tube 6.

### DETAILED DESCRIPTION

The conduit 12 is adapted to enable the comlector 2 to be removed from the lumen 14 of the airway tube 6. Advantageously, the provision of a removable comlector provides the clinician with greater flexibility during a medical procedure. Advantageously, the removal of the comlector 2 from the airway device 4 allows another comlector to be connected to the airway tube, and/or allows for manipulation of the airway tube and/or insertion of another device through the airway tube.

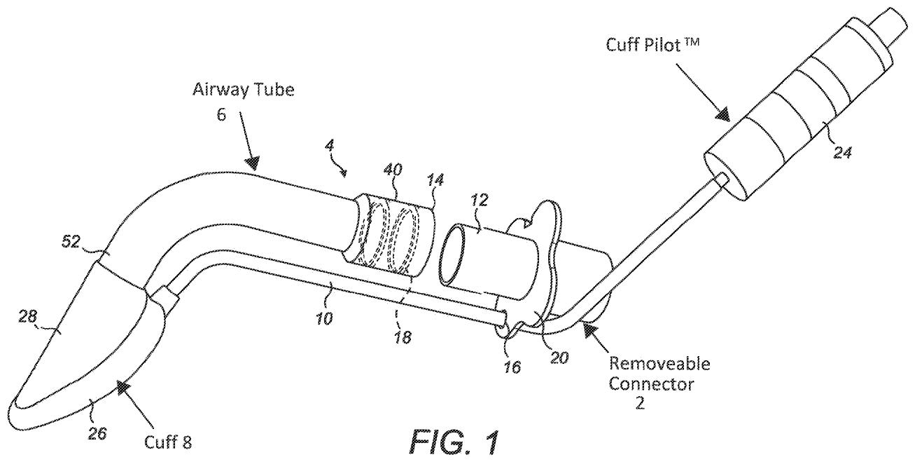

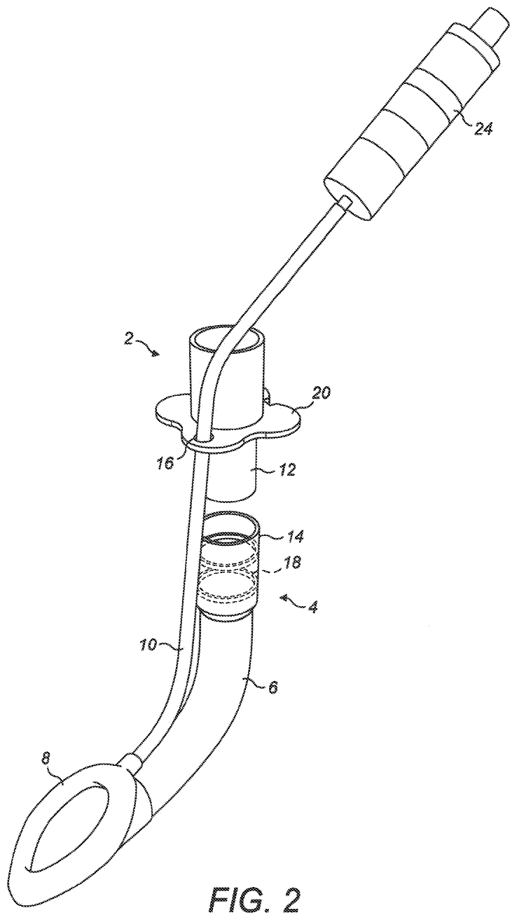

As shown in FIGS. 1 and 2, the conduit 12 of the connector 2 is adapted for removal from the lumen 14 of the airway tube 6 by having a friction fit with the internal surface of the lumen 14 of the airway tube 6. In this embodiment, the internal surface of the lumen 14 of the airway tube 6 comprises a plurality of circumferential friction reducing formations 18 which cooperate with the external surface of the conduit 12 of the connector 2. Advantageously, the friction reducing formations and/or other cooperating means and/or friction fit between the external surface of the conduit 12 of the connector and the internal surface of the airway tube lumen 14 allows the conduit 12 to be securely held in place within the airway tube 6 when the device is in use, and allows for removal of the connector 2 rom the airway tube 6 when required.

The conduit 12 of the connector 2 is adapted for removal from a preformed connector section 40 provided at the proximal end of the airway tube 6, wherein the prefonned connector section 40 of the airway tube is dimensioned to accommodate the connector 2. As shown, for example in FIG. 2, the preformed connector section of the airway tube 40 has a wider diameter than the diameter at the distal end 52 of the airway tube.

As shown, for example in FIG. 1, the distal end of the airway tube 2 has a substantially circular diameter, wherein the thickness of the walls at the top and bottom of the distal end of the airway tube is substantially the same as the thickness of the walls at the left and right of the distal end of the airway tube.

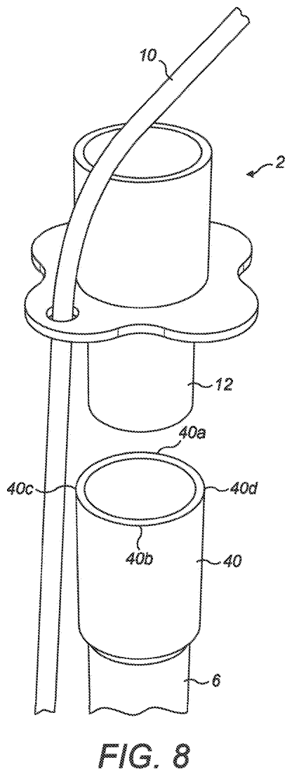

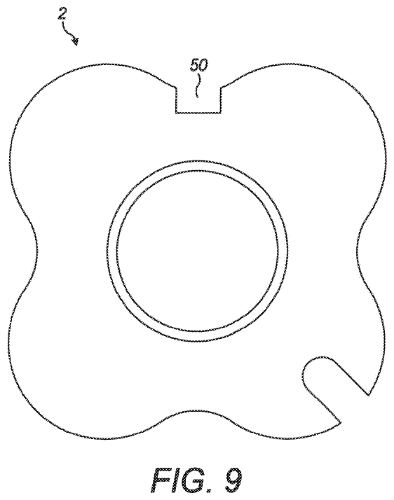

With reference to FIG. 8, the proximal end of the preformed connector section 40 of the airway tube has a substantially oval shape. Preferably, the thickness of the walls at the top 40a and bottom 40b of the preformed connector section of the airway tube is greater than the walls at the left 40c and right 40d of the preformed connector section of the airway tube. Advantageously, the oval shape of the preformed connector section of the airway tube assists in holding the connector securely within the airway tube, whilst allowing the connector to removed from the airway tube if required.

The connector 2 comprises means for tethering the connector 2 to an artificial airway device 4. The means for tethering comprises and inflation tube 10 which engages with a portion of the connector 2.

The connector 2 can move freely along the length of the inflation tube 10. The means for tethering allow the connector to move freely along the length of the inflation tube. Advantageously, this allows for greater flexibility during removal of the connector 2 from the airway tube 6. In this embodiment, the length of the inflation tube 10 is longer than the length of the airway tube 6 and the connector 2 can move along the length of the inflation tube 10 to assist in removal of the connector 2 from the airway tube 6.

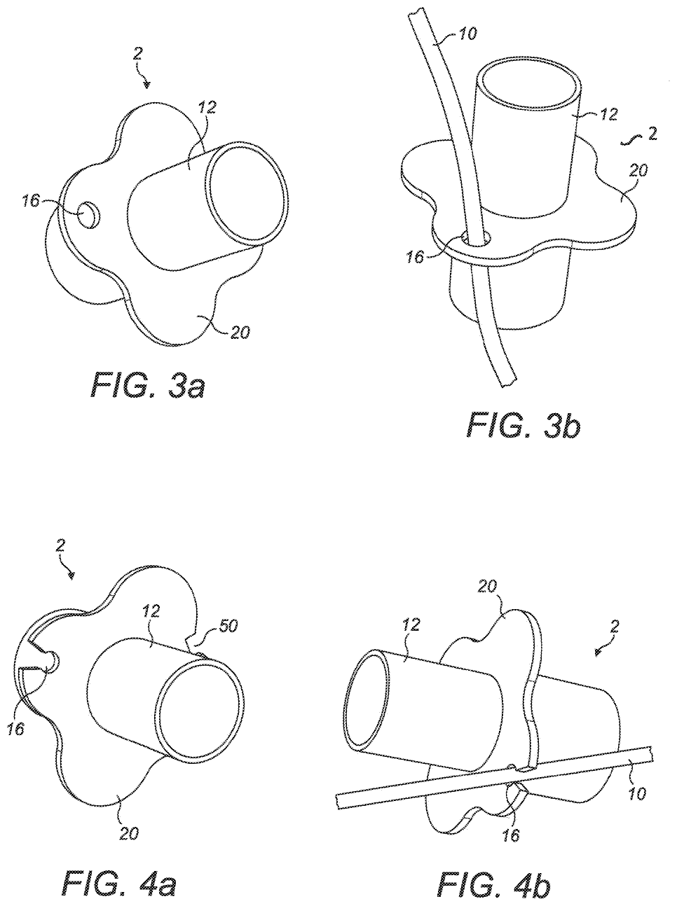

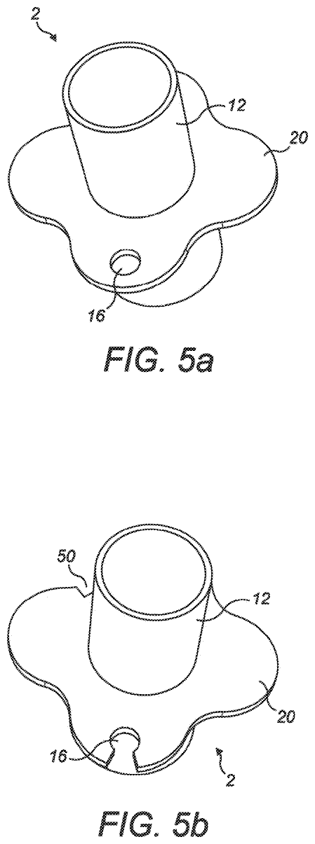

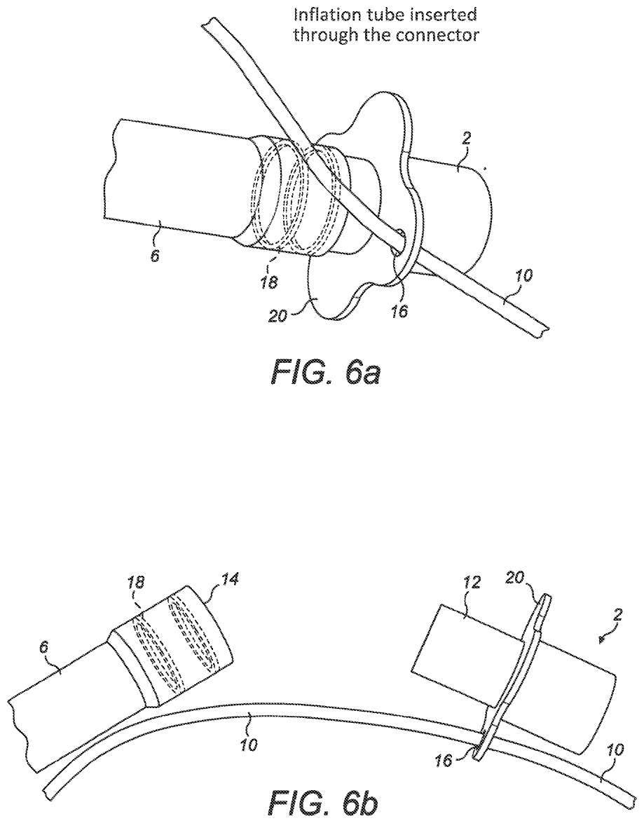

In the embodiment shown in FIGS. 3a, 3b, 5a, 6a and 6b, the means for tethering 16 holds the inflation tube 10 captive such that it is not detachable from the connector 2. Advantageously, this embodiment prevents the connector 2 from being inadvertently detached from the inflation tube 10 after being removed from the airway tube 6.

The connector 2 comprises a conduit 12 having a circumferential wall defining a lumen which may be inserted into the lumen 14 of the airway tube. The connector comprises a connector plate 20 which surrounds the conduit 12. The connector plate 20 extends from the conduit 12 at an angle substantially perpendicular to the axis of the conduit 12. The connector plate 20 comprises means for tethering 16 the inflation tube 10.

In the embodiment shown in FIGS. 3a, 3b, 5a, 6a and 6b, the means for tethering comprises at least one aperture 16 provided within the connector plate. In this embodiment, the at least one aperture 16 is a closed aperture that is provided within the connector plate 20. In this embodiment, the at least one aperture 16 is closed such that the inflation tube 10 is tethered to and held captive by the connector 2, such that the inflation tube 10 cannot be detached from the connector 2.

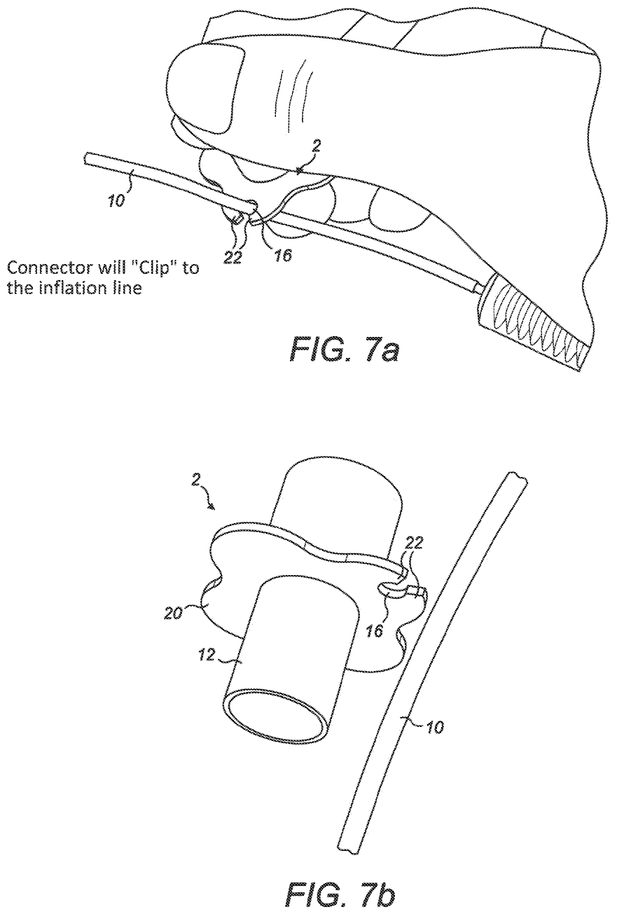

In another embodiment as shown in FIGS. 4a, 4b, 5b, 7a and 7b, the means for tethering 16 allows for attachment and/or detachment of the inflation tube 10 from the connector 2. Advantageously, this embodiment provides flexibility for the clinician if they need to use the connector during a medical procedure.

As shown in FIGS. 4a, 4b, 5b, 7a and 7b, the means for tethering 16 comprises a clip 22 that allows for the attachment and/or detachment of the inflation tube 10 from the connector 2. In this embodiment, the connector 2 comprises a clip 22 that allows for the attachment and/or detachment of the inflation tube 10 from the connector 2. In this embodiment, the clip 22 is provided on the connector plate 20. In this embodiment, the means for tethering comprises at least one aperture 16 that is open at one portion thereof, wherein the at least one open aperture 16 forms the clip 22 such that the inflation tube 10 may be selectively attached and/or detached from the connector 2. In this embodiment, the at least one open aperture 16 is provided on the connector plate 20.

The diameter of the at least one open or closed aperture on the connector and the diameter of the inflation tube are configured such that the inflation tube can freely slide within the at least one open or closed aperture. Preferably, the diameter of the open or closed aperture is between around 0.4 and 0.5 mm. Preferably, the diameter of the inflation tube is between around 0.2 and 0.3 mm.

As shown in FIGS. 1, 4a, 5b and 9, the connector plate comprises a recess 50. Preferably, the recess 50 is provided on a side of the connector plate that is opposite to the aperture 16. In one embodiment, the recess 50 may be provided on a side of the connector plate that is adjacent to the aperture 16. The recess is provided in the form of a cut away portion having three substantially straight sides. Advantageously, the recess assists in removal of the connector from the airway tube.

Typically, the cuff 8 is inflatable. Preferably, the inflation tube is attached to means 24 for monitoring and/or controlling the pressure within the cuff. In another embodiment, the cuff may be non-inflatable. In the embodiment shown in FIGS. 1 and 2, the inflation tube 10 is connected to the Cuff Pilot™_

The inflation tube 10 comprises a cuff pressure monitor or pilot valve that may be released from the inflation tube. Advantageously, the cuff pressure monitor or pilot valve may be released from the inflation tube without loss of cuff pressure.

Typically, the connector 2 is connected to a separate biteblock. In another embodiment, the connector comprises an integral biteblock. Preferably, the conduit of the connector may comprise a biteblock.

Preferably, the inner conduit of the connector (i.e. the part that is inserted into an airway tube) has a constant diameter. Preferably, the diameter of the inner conduit of the connector is between around 0.8 and 1.2 cm. More preferably, the diameter of the inner conduit of the connector is around 1.0 cm.

Typically, the connector 2 comprises a plastics material. Preferably, the connector 2 comprises PVC.

In one embodiment, the artificial airway device 4 is a laryngeal mask airway device. In one embodiment, the artificial airway device 4 is a laryngeal mask airway device.

With reference to FIGS. 1 and 2, there is provided an artificial airway device 4 to facilitate lung ventilation of a patient, comprising an airway tube 6 including a lumen 14, a mask 26 at one end of the airway tube, the mask including a backplate 28 and having a cuff 8 capable of forming a seal around the circumference of the laryngeal inlet, the cuff 8 surrounding a hollow interior space or lumen 30 of the mask and the airway tube 6 opening into the lumen of the mask, and an inflation tube 10 for selectively inflating and/or deflating the cuff 8, and a removable connector 2 disposed at the proximal end of the airway tube 6, the connector 2 comprising a conduit 12 which may be inserted into a lumen 14 of the airway tube 6, the connector 2 being adapted to be removable and replaceable from the lumen 14 of the airway tube 6.

The connector 2 comprises a conduit 12 which may be inserted into a lumen 14 of the airway tube 6. The conduit 12 may be adapted to enable the connector 2 to be removed from the lumen 14 of the airway tube 6. Advantageously, the provision of a removable connector 2 provides the clinician with greater flexibility during a medical procedure. Advantageously, the removal of the conduit 12 from the airway device 4 allows another connector to be connected to the airway tube, and/or allows for manipulation of the airway tube and/or insertion of another device through the airway tube.

As shown in FIGS. 1 and 2, the device 4 comprises means for tethering 16 the connector 2 to the artificial airway device 4. The means for tethering comprises an inflation tube 10 which engages with a portion of the connector 2.

As shown in FIGS. 1 and 2, the connector 2 can move freely along the length of the inflation tube 10. Preferably, the means for tethering allow the connector to move freely along the length of the inflation tube. Advantageously, this allows for greater flexibility during removal of the connector 2 from the airway tube 6.

Typically, the cuff 8 is inflatable. Preferably, the inflation tube 10 is attached to means for monitoring and/or controlling the pressure within the cuff. In one embodiment, the inflation tube is connected to a one way valve, as is known in the prior art. In the embodiment shown in FIGS. 1 and 2, the inflation tube is attached to the Cuff Pilot™.

The inflation tube 10 may include a cuff pressure monitor (48) or pilot valve that may be released from the inflation tube. Advantageously, the cuff pressure monitor or pilot valve may be released from the inflation tube without loss of cuff pressure.

Typically, the device 4 comprises a biteblock that is separate to the connector 2. In another embodiment, the device 4 comprises a biteblock that is integral with the connector 2. Preferably, the conduit 12 of the connector may comprise a biteblock.

The conduit 12 of the connector (i.e. the part that is inserted into an airway tube) has a constant diameter. Preferably, the diameter of the conduit of the connector is between around 0.8 and 1.2 cm. More preferably, the diameter of the conduit of the connector is around 1.0 cm.

Preferably, the artificial airway device 4 comprises a plastics material, such as PVC and/or silicone. Preferably, the connector 2 comprises PVC. Preferably, the inflation tube 10 comprises silicone.

In one embodiment, the artificial airway device 4 is a laryngeal mask airway device. In one embodiment, the artificial airway device 4 is a laryngeal mask airway device.

With reference to FIGS. 1 and 2, there is provided a method of using an artificial airway device 4 according to the second aspect of the invention comprising a removable connector 2 according to the first aspect of the invention, wherein the connector 2 is tethered to an inflation tube 10.

With reference to FIGS. 1 and 2, there is provided a method of treating a patient using an artificial airway device according to the second aspect of the invention.

In use, the artificial airway device 4 is inserted into a patient to establish an airway. In the assembled condition, the conduit 12 of the connector 2 is inserted into the lumen 14 of the airway tube 6 and held in place by means of a friction fit, wherein the external surface of the conduit of the connector engages with a plurality of circumferential formations 18 formed on the inner surface of the lumen 14 of the airway tube 6, at the proximal end thereof. In the assembled condition, the connector 2 is held securely within the airway tube 6 and may not accidentally be removed therefrom. In contrast with prior art devices, the connector 2 may be removed from the lumen 14 of the airway tube 6 by manipulating and/or pulling the connector 2 to release it from the lumen 14 of the airway tube 6. Due to the friction fit between the conduit 12 of the connector 2 and the lumen 14 of the airway tube 6, the removal of the connector 2 may be accomplished with minimal effort by the clinician. The removal of the connector 2 from the airway tube 6 provides the clinician with greater flexibility, allowing for example, an alternative comlector to be used with the airway device, and/or allowing the clinician access to the airway tube of the airway device.

The conduit 12 of the connector 2 may be adapted for removal from a preformed connector section 40 provided at the proximal end of the airway tube.

The connector 2 comprises means for tethering 16 the connector 2 to the artificial airway device 4. This is advantageous as it allows the connector to remain with the artificial airway device both when the connector 2 is attached to the airway tube 6 and when the connector 2 has been removed from the airway tube 6. Advantageously, this prevents accidental loss of the connector during a procedure, wherein the connector has been temporarily removed from the airway tube and will be reattached to the airway tube at a later stage in the procedure.

With reference to the drawings, the means for tethering the connector to the airway device 4 comprises an inflation tube 10 which engages with a portion of the connector 2.

In one embodiment, the means for tethering the connector to the airway device comprises a closed aperture 16 within the connector plate 20. In this embodiment, the inflation tube 10 is held captive within the connector plate 20 and cannot be detached therefrom. This embodiment has the advantage that the connector 2 is not accidentally detached from the inflation tube 10 during a procedure.

The diameter of the at least one open or closed aperture on the connector and the diameter of the inflation tube are configured such that the inflation tube can freely slide within the at least one open or closed aperture.

In another embodiment, the means for tethering the connector to the airway device comprises an open aperture 16 which may be provided in the form of a clip 22 within the connector plate 20. In this embodiment, the inflation tube 10 may be attached and/or detached from the connector 2, providing greater flexibility for the clinician.

The inflation tube 10 has a length that is longer than that of the airway tube 6. The connector may move freely along the length of the inflation tube 10. Advantageously, this assists in the removal of the connector 2 from the airway tube 6 while the inflation tube 10 is tethered to the connector 2.

1. An artificial airway device to facilitate lung ventilation of a patient, comprising:
an airway tube including a lumen, a mask at one end of the airway tube, the mask including a backplate and having a cuff capable of forming a seal around the circumference of the laryngeal inlet, the cuff surrounding a hollow interior space or lumen of the mask and the airway tube opening into the lumen of the mask, and
an inflation tube for selectively inflating and/or deflating the cuff, and
a removable connector disposed at the proximal end of the airway tube, the connector comprising a conduit adapted to be inserted into a lumen of the airway tube.

2. The device according to claim 1, wherein an external surface of the conduit comprises a screw thread which cooperates with a corresponding thread on an internal surface of the airway tube.

3. The device according to claim 1, wherein an external surface of the conduit cooperates with an internal surface of the airway tube by a push fit or friction fit with the airway tube.

4. The device according to claim 1, wherein the connector comprises a connector plate which surrounds the conduit.

5. The device according to claim 4, wherein the connector plate extends from the conduit at an angle substantially perpendicular to the axis of the conduit.

6. The device according to claim 4, wherein the connector comprises means for tethering the connector to the artificial airway device.

7. The device according to claim 6, wherein the connector is adapted to move freely along the length of the inflation tube, when the connector is removed from the airway tube.

8. The device according to claim 7, wherein the means for tethering comprises the inflation tube which engages with a portion of the connector.

9. The device according to claim 6, wherein the means for tethering comprises at least one aperture provided within the connector plate.

10. The device according to claim 9, wherein the means for tethering holds the inflation tube captive such that it is not removable from the connector.

11. The device according to claim 10, wherein the at least one aperture is a closed aperture that is provided within the material of the connector plate.

12. The device according to claim 9, wherein the means for tethering allows for attachment and/or detachment of the inflation tube from the connector.

13. The device according to claim 12, wherein the at least one aperture is an open aperture that is provided within the material of the connector plate.

14. The device according to claim 6, wherein the connector comprises a biteblock that is integral with the connector.

15. The device according to claim 6, wherein the inflation tube comprises a cuff pressure monitor or pilot valve that is releasable from the inflation tube.

16. The device according to claim 6, wherein an external surface of the conduit comprises a screw thread which cooperates with a corresponding thread on an internal surface of the airway tube.

17. The device according to claim 6, wherein an external surface of the conduit cooperates with an internal surface of the airway tube by a push fit or friction fit with the airway tube.

18. The device according to claim 1, wherein the connector comprises a biteblock that is integral with the connector.

19. The device according to claim 1, wherein the inflation tube comprises a cuff pressure monitor or pilot valve that is releasable from the inflation tube.

20. The device according to claim 6, wherein the connector plate extends from the conduit at an angle substantially perpendicular to the axis of the conduit, and wherein the means for tethering comprises at least one aperture provided within the connector plate, and wherein the connector is adapted to move freely along the length of the inflation tube, when the connector is removed from the airway tube.
